(19) United States
(12) Patent Application Publication
(10) Pub. No.: US 2026/0176566 A1
(43) Pub. Date: Jun. 25, 2026

(54) CYCLING FERMENTATION, PROCESS AND SYSTEM FOR SUBMERGED FERMENTATION

(71) Applicant: Peter Dean, Scotchtown (CA)

(72) Inventor: Peter Dean, Scotchtown (CA)

(21) Appl. No.: 18/842,781

(22) PCT Filed: May 24, 2024
(86) PCT No.: PCT/CA2024/050692

§ 371 Date: Aug. 29, 2024

Related U.S. Application Data
(60) Provisional application No. 63/468,647, filed on May 24, 2023.

(51) Int. Cl. C12M 1/00; C12M 1/34

(52) U.S. Cl. CPC C12M 29/06; C12M 23/40; C12M 23/58; C12M 29/20; C12M 41/34; C12M 41/44

(57) ABSTRACT

Fermentation processes are described herein. The processes include a) supplying a first fermentation tank with a fermentation broth; b) supplying gas from an external gas source to the first fermentation tank and to a second fermentation tank; c) during step b), venting gas from the second fermentation tank into the first fermentation tank via a fluid pathway, and venting gas from the first fermentation tank via a vent of the first fermentation tank; and d) during step b) and after step c), closing the vent of the first fermentation tank, and opening a vent of the second fermentation tank, to force the fermentation broth from the first fermentation tank into the second fermentation tank via the fluid pathway.

### CROSS-REFERENCE

The present application claims priority to U.S. Provisional Patent Application No. 63/468,647 titled Cycling Fermentation, Process and System for Submerged Fermentation filed on May 24, 2023, the entire contents of which are hereby incorporated by reference herein.

### FIELD

The present application relates to fermentation processes, and more specifically, to submerged fermentation as it relates to processes and systems for the production of biomass and associated metabolites.

### BACKGROUND

U.S. Patent Application Publication No. US2005/0146982 (Carlson et al.) discloses an apparatus and method for measuring and mixing liquids. First and second vessels are connected to an aspirator so that reduced pressure is produced in the second vessel as a first liquid flows to the first vessel. Reduced pressure motivates flow of a second liquid into the second vessel until a predetermined level is reached. A conduit opens between the first and second vessels. The aspirator draws liquid from the second vessel to the first vessel while the conduit allows liquid from the first vessel to flow to the second vessel. Thus, the liquids are mixed until a predetermined level of liquid in the first vessel is reached.

U.S. Pat. No. 4,449,827 (Karkiewicz) discloses a device for thoroughly mixing the liquid contents of first and second containers without exposing either the liquid contents or the resultant mixture to outside contamination. The device features a movable pumping diaphragm which traverses a pumping chamber so that the pumping chamber is divided into an upper and lower chamber. In a first position, the diaphragm causes the upper chamber to have a reduced volume while the lower chamber has an increased volume. In a second position, the pumping diaphragm causes the lower chamber to have a decreased volume while the upper chamber has an increased volume. Valving assemblies are utilized in conjunction with both the upper and lower chamber to selectively allow for the ingress and egress of fluids from these chambers dependent upon the position of the pumping diaphragm.

### SUMMARY

The following summary is intended to introduce the reader to various aspects of the disclosure, but not to define or delimit any invention.

According to some aspects, a fermentation process is described herein. The process includes a) supplying a first fermentation tank with a fermentation broth; b) supplying gas from an external gas source to the first fermentation tank and to a second fermentation tank; c) during step b), venting gas from the second fermentation tank into the first fermentation tank via a fluid pathway, and venting gas from the first fermentation tank via a vent of the first fermentation tank; and d) during step b) and after step c), closing the vent of the first fermentation tank, and opening a vent of the second fermentation tank, to force the fermentation broth from the first fermentation tank into the second fermentation tank via the fluid pathway.

In some embodiments, step d) includes forcing substantially all of the fermentation broth from the first fermentation tank into the second fermentation tank via the fluid pathway.

In some embodiments, the process further includes: e) during step b) and after step d), closing the vent of the second fermentation tank, and opening the vent of the first fermentation tank, to force the fermentation broth from the second fermentation tank into the first fermentation tank via the fluid pathway. Steps d) and e) can be repeated to repeatedly transfer the fermentation broth between the first fermentation tank and the second fermentation tank. Step e) can include forcing substantially all of the fermentation broth from the second fermentation tank into the first fermentation tank via the fluid pathway.

In some embodiments, step b) includes supplying compressed air from a compressed air source to the first fermentation tank and to the second fermentation tank.

In some embodiments, step b) includes supplying gas to the first fermentation tank via a gas inlet at a lower portion of the first fermentation tank. In some embodiments, step b) includes supplying gas to the second fermentation tank via a gas inlet at a lower portion of the second fermentation tank.

In some embodiments, step c) includes venting gas through a bottom port of the second fermentation tank into a bottom port of the first fermentation tank, via the fluid pathway. Step c) can include venting gas from an upper portion of the first fermentation tank via the vent of the first fermentation tank. Step d) can include forcing the fermentation broth through the bottom port of the first fermentation tank into the bottom port of the second fermentation tank via the fluid pathway. Step d) can include venting gas from an upper portion of the second fermentation tank via the vent of the second fermentation tank.

In some embodiments, step b) further includes supplying gas from the external gas source to a third fermentation tank. Step c) can further include: during step b), venting gas from the second fermentation tank into the third fermentation tank via the fluid pathway, and venting gas from the third fermentation tank via a vent of the third fermentation tank. The process can further include: e) during step b) and after step d), closing the vent of the second fermentation tank, and opening the vent of the third fermentation tank, to force the fermentation broth from the second fermentation tank into the third fermentation tank via the fluid pathway.

In some embodiments, step b) further includes supplying gas from the external gas source to a fourth fermentation tank. Step c) can further include: during step b), venting gas from the second fermentation tank into the fourth fermentation tank via the fluid pathway, and venting gas from the fourth fermentation tank via a vent of the fourth fermentation tank. The process can further include: f) during step b) and after step e), closing the vent of the third fermentation tank, and opening the vent of the fourth fermentation tank, to force the fermentation broth from the third fermentation tank into the fourth fermentation tank via the fluid pathway.

According to some aspects, a process for fermentation includes: a) supplying a first fermentation tank with a fermentation broth; b) supplying gas from an external gas source to a gas inlet at a lower portion of the first fermentation tank and to a gas inlet at a lower portion of a second fermentation tank; c) during step b), venting gas from a bottom port of the second fermentation tank into a bottom port of the first fermentation tank via a fluid pathway, and venting gas from the first fermentation tank via a vent at an upper portion of the first fermentation tank; d) during step b) and after step c), closing the vent of the first fermentation tank, and opening a vent at an upper portion of the second fermentation tank, to force substantially all of the fermentation broth from the first fermentation tank into the second fermentation tank, via the bottom port of the first fermentation tank, the fluid pathway, and the bottom port of the second fermentation tank; and e) during step b) and after step d), closing the vent of the second fermentation tank, and opening the vent of the first fermentation tank, to force substantially all of the fermentation broth from the second fermentation tank into the first fermentation tank, via the bottom port of the second fermentation tank, the fluid pathway, and the bottom port of the first fermentation tank.

According to some aspects, a system for fermentation includes a first fermentation tank and a second fermentation tank. Each fermentation tank has, respectively, a lower portion with a gas inlet and a bottom port, and an upper portion with an openable and closeable vent. An external gas source is in communication with the gas inlet of the first fermentation tank and the gas inlet of the second fermentation tank, for supplying gas to the first fermentation tank and the second fermentation tank. A fluid pathway extends from the bottom port of the first fermentation tank to the bottom port of the second fermentation tank, for providing fluid communication between the first fermentation tank and the second fermentation tank.

In some embodiments, the system further comprises a first sparger in the first fermentation tank and serving as the gas inlet of the first fermentation tank, and a second sparger in the second fermentation tank and serving as the gas inlet of the second fermentation tank.

In some embodiments, the vent of the first fermentation tank is at an uppermost portion of the first fermentation tank, and the vent of the second fermentation tank is at an uppermost portion of the second fermentation tank.

In some embodiments, the bottom port of the first fermentation tank is at a lowermost portion of the first fermentation tank, and the bottom port of the second fermentation tank is at a lowermost portion of the second fermentation tank.

In some embodiments, the external gas source includes a compressed air source.

In some embodiments, the system further includes a third fermentation tank. The external gas source and the fluid pathway can be in communication with the third fermentation tank. In some embodiments, the system further includes fourth fermentation tank. The external gas source and the fluid pathway can be in communication with the fourth fermentation tank. The system can further include a set of valves for controlling flow amongst the first, second, third and fourth fermentation tanks via the fluid pathway.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

The drawings included herewith are for illustrating various embodiments of articles, methods, and apparatuses of the present specification and are not intended to limit the scope of what is taught in any way. In the drawings:

FIG. 1 is a schematic view of an embodiment of a system for fermentation, wherein a fermentation broth is in a first fermentation tank;

FIG. 2 is a schematic view of the system of FIG. 1, wherein the fermentation broth is in the process of being transferred to a second fermentation tank;

FIG. 3 is a schematic view of the system of FIG. 1, wherein substantially all of the fermentation broth has been transferred to the second fermentation tank;

FIG. 4 is a schematic view of another embodiment of a system for fermentation, wherein first, third and fourth fermentation tanks contain a fermentation broth, and a second fermentation tank is generally empty;

FIG. 5 is a schematic view of the system of FIG. 1, wherein substantially all of the fermentation broth from the first fermentation tank has been transferred to the second fermentation tank;

FIG. 6 is a schematic view of the system of FIG. 1, wherein substantially all of the fermentation broth from the third fermentation tank has been transferred to the first fermentation tank; and

FIG. 7 is a schematic view of another embodiment of a system for fermentation, wherein first, third and fourth fermentation tanks contain a fermentation broth, and a second fermentation tank is generally empty.

### DETAILED DESCRIPTION

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Disclosed herein are fermentation processes and systems. The systems generally include two or more fermentation tanks (i.e., a first fermentation tank, a second fermentation tank, and optionally subsequent fermentation tanks), in which fermentation can take place. The fermentation tanks can be in fluid communication via a fluid pathway, and an external gas source can supply gas to the fermentation tanks. In some embodiments, in use, one or more of the fermentation tanks contains a fermentation broth (e.g., the first fermentation tank), and one or more of the fermentation tanks (e.g., the second fermentation tank) remains generally empty (i.e., is not filled with fermentation broth). Upon completion of a fermentation cycle in the first fermentation tank, a pressure differential is generated using the external gas source, to transfer the fermentation broth from the first fermentation tank to the second fermentation tank, via the fluid pathway. A second fermentation cycle can then begin in the second fermentation tank. Upon completion of the second fermentation cycle, a second pressure differential is generated using the external gas source, to transfer the fermentation broth from the second fermentation tank back to the first fermentation tank, or to a third or subsequent fermentation tank. This transferring of the fermentation broth can be repeated generally continuously, for example at set time intervals, when a fermentation milestone has been reached, or when deemed necessary or desirable.

The transferring of the fermentation broth from one fermentation tank to another can affect low shear mixing of the fermentation broth. This mixing can be achieved without the need for mechanical agitation (e.g., without the need for an impeller), which can in turn reduce the potential for contamination of the fermentation broth, reduced energy s and reduce cleaning time. Furthermore, the fermentation broth can be transferred out of one fermentation tank and into another fermentation tank via bottom ports of the fermentation tanks. This can result in a high degree of mixing, since buoyant solids that have collected at the surface of the one tank are forced to enter the bottom of the other tank. Furthermore, denser solids that have collected at the bottom of the one tank are carried with the liquid flow towards the top of the other tank. This may be particularly beneficial in submerged fermentation processes for the production of fungal biomass. Fungal biomass includes mycelium that tends to adhere to internal components of fermentation tanks, including but not limited to impellers and/or other components for mechanical mixing.

The transferring of the fermentation broth from one fermentation tank to another can also affect scaling issues of the systems and processes described herein. By having two or more fermentation tanks, a height of the tanks can be reduced when compared to similar systems and processes that have a single tank without reducing a total volume of the system.

As used herein, the term ‘fermentation broth’ includes any liquid in which fermentation is taking place or has taken place, or any liquid in which fermentation is intended to take place. For example, the term ‘fermentation broth’ may refer to a liquid mixture of water, nutrients, and biomass.

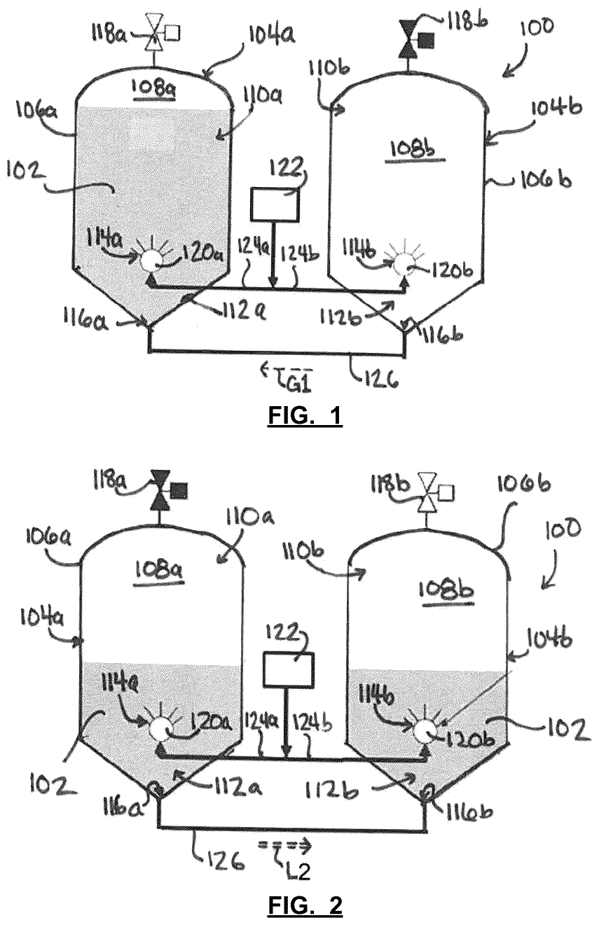

Referring now to FIG. 1, an embodiment of a system 100 for fermentation is shown. The system 100 includes a first fermentation tank 104a, and a second fermentation tank 104b. In FIG. 1, the first fermentation tank 104a contains a fermentation broth, while the second fermentation tank is generally empty.

In addition to the components described below, the fermentation tanks can include various other components such as valves, sensors, ports, indicators, and heating/cooling mechanisms, which for simplicity are not described herein.

In the embodiment shown, the first fermentation tank has a tank wall 106a defining a tank volume 108a. The tank volume 108a can be, for example, between 10 litres and 10,000 litres, or between 10,000 litres and 100,000 litres. Similarly, in the embodiment shown, the second fermentation tank 104b has a tank wall 106b defining a tank volume 108b. The tank volume 108b can be, for example, between 10 litres and 10,000 litres, or between 10,000 litres and 100,000. The first 104a and second 104b fermentation tanks can be of the same volume, or of a different volume.

In the embodiment shown, the first fermentation tank 104a further has an upper portion 110a, and a lower portion 112a. The lower portion 112a includes a gas inlet 114a, and a bottom port 116a. In the embodiment shown, the bottom port 116a is at a lowermost portion of the first fermentation tank 104a, to facilitate the removal of substantially all of the fermentation broth 102 from the first fermentation tank 104a. The upper portion 110a includes an openable and closeable vent 118a. In the embodiment shown, the vent 118a is at an uppermost portion of the first fermentation tank 104a.

Similarly, in the embodiment shown, the second fermentation tank 104b has an upper portion 110b, and a lower portion 112b. The lower portion 112b includes a gas inlet 114b, and a bottom port 116b. In the embodiment shown, the bottom port 116b is at a lowermost portion of the second fermentation tank 104b. The upper portion 110b includes an openable and closeable vent 118b. In the embodiment shown, the vent 118b is at an uppermost portion of the second fermentation tank 104a.

In the embodiment shown, each fermentation tank 104a, 104b includes a sparger 120a, 120b, respectively. The sparger 120a serves as the gas inlet 114a of the first fermentation tank 104a, and the sparger 120b serves as the gas inlet 114b of the second fermentation tank 104b. The spargers 120a, 120b are both in communication with an external gas source 122, via gas lines 124a, 124b, respectively. The external gas source 122 can supply a gas such as air or oxygen to the fermentation tanks 104a, 104b. For example, the external gas source can include a compressed air source.

In the embodiment shown, a fluid pathway 126 provides fluid communication between the first fermentation tank 104a and the second fermentation tank 104b. The fluid pathway extends from the bottom port 116a of the first fermentation tank 104a to the bottom port 116b of the second fermentation tank. The fluid pathway 126 can include one or more pipes or conduits. As will be described below with regards to FIG. 7, the fluid pathway 126 can optionally include valves for controlling the flow between the fermentation tanks 104a, 104b. The fluid pathway can also optionally include various other components such as valves, sensors, ports, indicators, and heating/cooling mechanisms, which for simplicity are not described herein.

The system 100 may be used in various types of fermentation, such as but not limited to fermentation with a long filamentous fungus (e.g., Rhizopus oryzae) for the production of fungal biomass and associated metabolites, such as but not limited to the production of chitosan (which can be extracted from the produced fungal biomass). It should be understood that many other commercially viable species could also be used in the processes and systems described herein, including but not limited to shear-sensitive organisms that could be harmed by mechanical mixers. An embodiment of a process for using the system 100 for fermentation will be described below; however, the system 100 may be used according to other processes, and the process may be carried out with other systems. Furthermore, for simplicity, details of the fermentation process, such as instrumentation and control equipment necessary for data collection, monitoring and control of liquid levels, temperature, pressure and flow conditions and components of growth media, will not be described herein.

Referring still to FIG. 1, in the embodiment shown, fermentation is carried out in the first fermentation tank 104a, while the second fermentation tank 104b remains generally empty. For example, the fermentation broth 102 may be supplied to the first fermentation tank 104a, and fermentation may be carried out in the first fermentation tank 104a for a period of time. The period of time may be, for example, between zero seconds and 10 minutes, or between 10 minutes and 60 days. This period of fermentation in the first fermentation tank 104a may be referred to as a ‘first fermentation cycle’.

Referring still to FIG. 1, in the embodiment shown, during the first fermentation cycle, gas is supplied from the external gas source 122 to both the first fermentation tank 104a and the second fermentation tank 104b. As mentioned above, the gas may be compressed air, and flows from a compressed air source along gas lines 124a, 124b, to the respective spargers 120a, 120b in the lower portions 112a, 112b of the fermentation tanks 104a, 104b. Furthermore, during the first fermentation cycle, the vent 118a of the first fermentation tank 104a is open, and the vent 118b of the second fermentation tank 104b is closed. In addition, any valves along the fluid pathway 126 are open, so that the first fermentation tank 104a and second fermentation tank 104b are in communication via the fluid pathway 126. Accordingly, gas supplied to the second fermentation tank 104b is vented into the first fermentation tank 104a via the bottom port 116b of the second fermentation tank 104b, the fluid pathway 126, and the bottom port 116a of the first fermentation tank 104a. The gas then bubbles through the fermentation broth to the upper portion 110a of the first fermentation tank 104a. Any gas supplied to the first fermentation tank 104a (i.e., directly from the external gas source 122 and from the external gas source 122 via the second fermentation tank 104b), is vented from the upper portion 110a of the first fermentation tank 104a via the vent 118a in the upper portion 110a of the first fermentation tank 104a. In FIG. 1, the flow of gas is indicated by arrow G1. The flow of gas from the second fermentation tank 104b into the first fermentation tank 104b via the bottom port 116a of the first fermentation tank 104a generally prevents the fermentation broth from draining from the first fermentation tank 104a via the bottom port 116a.

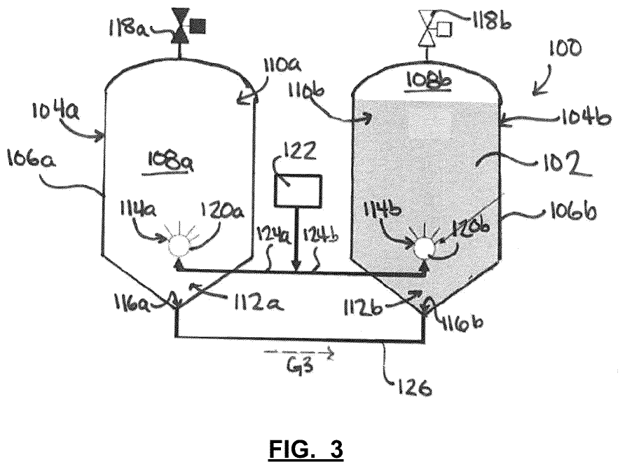

Referring now to FIG. 2, in the embodiment shown, when the first fermentation cycle is complete (e.g. when a set period of time has passed, when a fermentation milestone has been reached, or when mixing or fluid transfer is deemed necessary or desirable) the supply of gas to the fermentation tanks 104a, 104b is continued, the vent 118a of the first fermentation tank 104a is closed, and the vent 118b of the second fermentation tank 104b is opened. This causes pressure to increase in the first fermentation tank 104a and to decrease in the second fermentation tank 104b, which forces the fermentation broth 102 to flow from the first fermentation tank 104a into the second fermentation tank 104b via the bottom port 116a of the first fermentation tank 104a, the fluid pathway 126, and the bottom port 116b of the second fermentation tank. In FIG. 2, the flow of liquid is indicated by arrows L2.

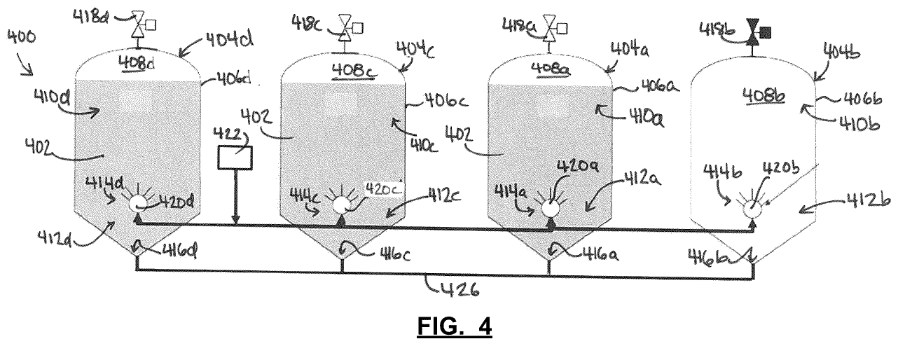

In the embodiment shown, the bottom ports 116a, 116b are at the lowermost portions of the fermentation tanks 104a, 104b. As such, as shown in FIG. 3, substantially all of the fermentation broth 102 is forced from the first fermentation tank 104a into the second fermentation tank 104b, via the fluid pathway 126.

In the embodiment shown, the transfer of the fermentation broth 102 from the first fermentation tank 104a to the second fermentation tank 104b results in mixing of the fermentation broth 102. For example, buoyant solids that may have collected at the surface of the liquid during the first fermentation cycle will enter the bottom port 116b of the second fermentation tank 104b, and flow upwards through the second fermentation tank 104b. Furthermore, denser solids that may have collected at the bottom of the first fermentation tank 104a during the first fermentation cycle may be carried towards the top of the second fermentation tank 104b by the flow of the liquid.

Referring now to FIG. 3, in the embodiment shown, after substantially all of the fermentation broth 102 is forced from the first fermentation tank 104a into the second fermentation tank 104b, a second fermentation cycle begins in the second fermentation tank 104b. During the second fermentation cycle, the vent 118a of the first fermentation tank 104a remains closed, and the vent 118b of the second fermentation tank 104b remains open. Further, the supply of gas from the external gas source 122 is continued. Gas supplied to the first fermentation tank 104a is vented into the second fermentation tank 104b via the fluid pathway 126, and then bubbles through the fermentation broth 102 to the upper portion 110a of the second fermentation tank 104b. Furthermore, any gas supplied to the second fermentation tank 104b (i.e., directly from the external gas source 122 and from the external gas source 122 via the first fermentation tank 104a), is vented from the second fermentation tank 104b via the vent 118b of the second fermentation tank 104b. In FIG. 3, the flow of gas is indicated by arrow G3.

When second fermentation cycle is complete (e.g., when a set period of time has passed, when a fermentation milestone has been reached, or when mixing is deemed necessary or desirable), the fermentation broth 102 may be forced back to the first fermentation tank 104a, so that the first fermentation cycle can begin again. Particularly, the supply of gas to the fermentation tanks 104a, 104b can be continued, the vent 118b of the second fermentation tank 104b can be closed, and the vent 118a of the first fermentation tank 104a can be opened. This causes pressure to increase in the second fermentation tank 104b and to decrease in the first fermentation tank 104a, which forces the fermentation broth 102 to flow from the second fermentation tank 104b into the first fermentation tank 104a via the fluid pathway 126. Again, substantially all of the fermentation broth 102 may be transferred from the second fermentation tank 104b to the first fermentation tank 104a.

The above process—i.e., the transfer of the fermentation broth between the first fermentation tank and the second fermentation tank—may be repeated continuously, to continuously mix the fermentation broth.

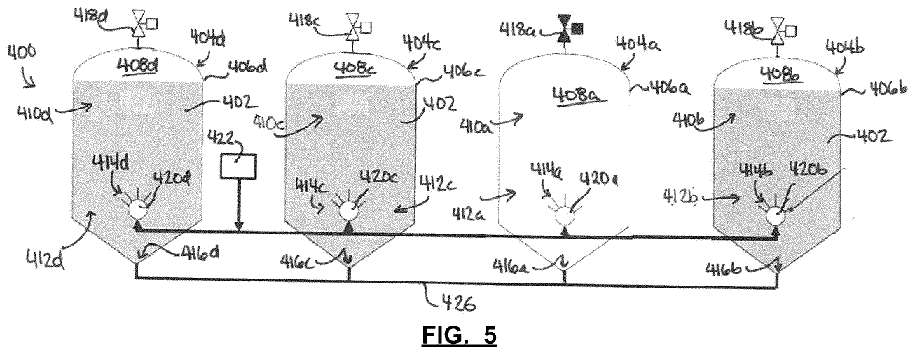

Referring now to FIGS. 4 to 6, another embodiment of a system for fermentation is shown. In FIGS. 4 to 6, like reference numerals as in FIGS. 1 to 3, incremented by 300, will be used.

In the embodiment shown, similarly to the system 100 of FIGS. 1 to 3, the system 400 includes first 404a and second 404b fermentation tanks. The system 400 further includes a third fermentation tank 404c and a fourth fermentation tank 404d. Each of the fermentation tanks 404a-404d includes, respectively, a tank wall 406a-406d defining a tank volume 408a-408d, an upper portion 410a-410d with a vent 418a-418d, and a lower portion 412a-412d with a bottom port 416a-416d and a sparger 420a-420d that provides a gas inlet 414a-414d.

In the embodiment shown, an external gas source 422 is in communication with the first through fourth fermentation tanks 404a-404d, and a fluid pathway 426 provides fluid communication amongst the first through fourth fermentation tanks 404a-404d via the bottom ports416a-416d.

Referring still to FIG. 4, in the embodiment shown, in a first fermentation cycle, the first 404a, third 404c, and fourth 404d fermentation tanks are supplied with a fermentation broth 402, while the second fermentation tank 404b remains empty. Fermentation is carried out in the first 404a, third 404c, and fourth 404d fermentation tanks for a period of time. The respective vents 418a, 418c, 418d of the first 404a, third 404c, and fourth 404d fermentation tanks are open during the first fermentation cycle, and the vent 418b of the second fermentation tank 404b is closed. Gas is supplied to the spargers of all four fermentation tanks 404a-404d. Because the vent 418b of the second fermentation tank 404b is closed, the gas flows from the second fermentation tank 404b into the first 404a, third 404c, and fourth 404d fermentation tanks via the fluid pathway 426, and is vented from the first 404a, third 404c, and fourth 404d fermentation tanks via the vents 418a, 418c, 418d.

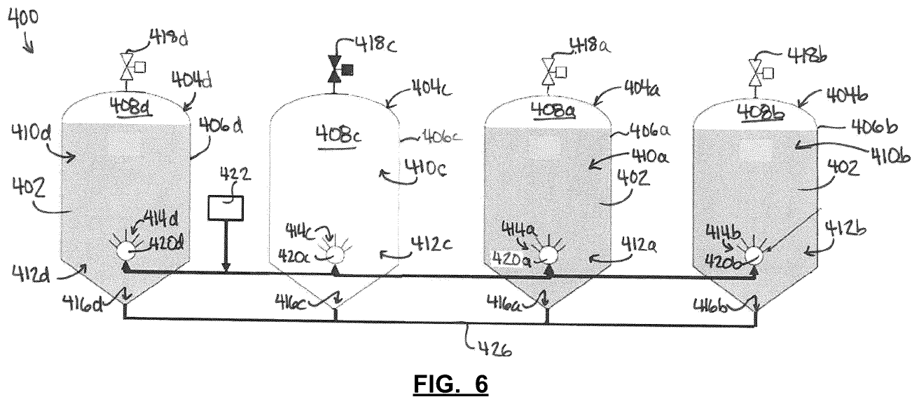

Referring now to FIG. 5, in the embodiment shown, when the first fermentation cycle is complete (e.g. when a set period of time has passed, when a fermentation milestone has been reached, or when mixing is deemed necessary or desirable) the supply of gas to the fermentation tanks 404a-404d is continued, the vent 418a of the first fermentation tank 404a is closed, and the vent 418b of the second fermentation tank 404b is opened. This causes pressure to increase in the first fermentation tank 404a and to decrease in the second fermentation tank 404b, which forces the fermentation broth 402 to flow from the first fermentation tank 404a into the second fermentation tank 404b via the bottom port 416a of the first fermentation tank 404a, the fluid pathway 426, and the bottom port 416b of the second fermentation tank 404b, as described above with respect to FIGS. 1 to 3. A second fermentation cycle then begins in the second fermentation tank 404b.

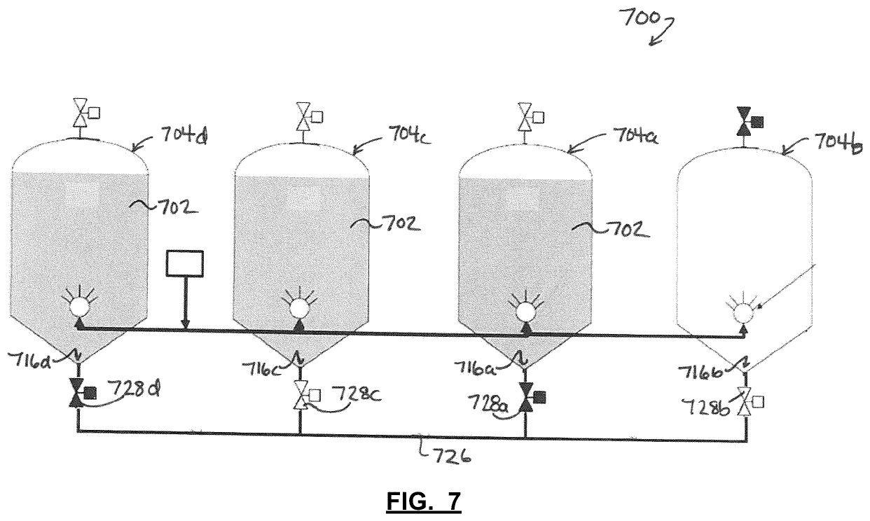

Referring now to FIG. 6, in the embodiment shown, when the second fermentation cycle is complete (e.g. when a set period of time has passed, when a fermentation milestone has been reached, or when mixing is deemed necessary or desirable) the supply of gas to the fermentation tanks 404a-404d is continued, the vent 418b of the second fermentation tank 404b is closed, and the vent 418c of the third fermentation tank 404c is opened. This causes pressure to increase in the second fermentation tank 404b and to decrease in the third fermentation tank 404c, which forces the fermentation broth 402 to flow from the second fermentation tank 404b into the third fermentation tank 404c via the bottom port 416b of the second fermentation tank 404b, the fluid pathway 426, and the bottom port 416c of the third fermentation tank 404c. A third fermentation cycle then begins in the third fermentation tank.

When the third fermentation cycle is complete, a fourth fermentation cycle, and subsequent fermentation cycles, can take place, by closing the vent of a given fermentation tank, and opening the vent of an empty fermentation tank, to transfer the fermentation broth 402 from tank to tank and thereby mix the fermentation broth.

While the system of FIGS. 4 to 6 uses four tanks, other embodiments may use another number of tanks (e.g., 3 tanks, or more than 4 tanks).

The system 400 of FIGS. 4 to 6, and related process, may be useful in scaling up a fermentation process. The use of several tanks can both allow for mixing and avoid the need for a single large tank. Furthermore, the fermentation tanks can be spaced out horizontally, which can be useful in situations where height is constricted.

Referring now to FIG. 7, another embodiment of a system for mixing a fermentation broth is shown. In FIG. 7 like reference numerals as in FIGS. 4 to 6, incremented by 300, will be used.

The system 700 is similar to the system 400; however, a set of valves is provided for controlling flow between the first 704a, second 704b, third 704c, and fourth 704d fermentation tanks via the fluid pathway 726. The set of valves includes a first valve 728a for controlling flow to and from the first fermentation tank 704a via the bottom port 716a of the first fermentation tank 704a, a second valve 728b for controlling flow to and from the second fermentation tank 704b via the bottom port 716b of the second fermentation tank 704b, a third valve 728c for controlling flow to and from the third fermentation tank 704c via the bottom port 716c of the third fermentation tank 704c, and a fourth valve 728dfor controlling flow to and from the fourth fermentation tank 704d via the bottom port 716d of the fourth fermentation tank 704d.

The system 700 can be operated in a similar fashion to the system 400; however the valves 728a-728d can be selectively opened and closed to direct or prevent flow of gas and fermentation broth 702 into a given fermentation tank.

While the above description provides embodiments of one or more processes or apparatuses, it will be appreciated that other processes or apparatuses may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

What is claimed is:

1. A fermentation process comprising:
a) supplying a first fermentation tank with a fermentation broth;
b) supplying gas from an external gas source to the first fermentation tank and to a second fermentation tank;
c) during step b), venting gas from the second fermentation tank into the first fermentation tank via a fluid pathway, and venting gas from the first fermentation tank via a vent of the first fermentation tank; and
d) during step b) and after step c), closing the vent of the first fermentation tank, and opening a vent of the second fermentation tank, to force the fermentation broth from the first fermentation tank into the second fermentation tank via the fluid pathway.

2. The process of claim 1, wherein step d) comprises forcing substantially all of the fermentation broth from the first fermentation tank into the second fermentation tank via the fluid pathway.

3. The process of claim 1 or claim 2, further comprising:
e) during step b) and after step d), closing the vent of the second fermentation tank, and opening the vent of the first fermentation tank, to force the fermentation broth from the second fermentation tank into the first fermentation tank via the fluid pathway.

4. The process of claim 3, further comprising repeating steps d) and e) to repeatedly transfer the fermentation broth between the first fermentation tank and the second fermentation tank.

5. The process of claim 3 or claim 4, wherein step e) comprises forcing substantially all of the fermentation broth from the second fermentation tank into the first fermentation tank via the fluid pathway.

6. The process of any one of claims 1 to 5, wherein step b) comprises supplying compressed air from a compressed air source to the first fermentation tank and to the second fermentation tank.

7. The process of any one of claims 1 to 6, wherein step b) comprises supplying gas to the first fermentation tank via a gas inlet at a lower portion of the first fermentation tank.

8. The process of any one of claims 1 to 7, wherein step b) comprises supplying gas to the second fermentation tank via a gas inlet at a lower portion of the second fermentation tank.

9. The process of any one of claims 1 to 8, wherein step c) comprises venting gas through a bottom port of the second fermentation tank into a bottom port of the first fermentation tank, via the fluid pathway.

10. The process of claim 9, wherein step c) comprises venting gas from an upper portion of the first fermentation tank via the vent of the first fermentation tank.

11. The process of claim 9 or claim 10, wherein step d) comprises forcing the fermentation broth through the bottom port of the first fermentation tank into the bottom port of the second fermentation tank via the fluid pathway.

12. The process of claim 11, wherein step d) comprises venting gas from an upper portion of the second fermentation tank via the vent of the second fermentation tank.

13. The process of claim 1, wherein step b) further comprises supplying gas from the external gas source to a third fermentation tank.

14. The process of claim 13, wherein step c) further comprises: during step b), venting gas from the second fermentation tank into the third fermentation tank via the fluid pathway, and venting gas from the third fermentation tank via a vent of the third fermentation tank.

15. The process of claim 14, further comprising:
e) during step b) and after step d), closing the vent of the second fermentation tank, and opening the vent of the third fermentation tank, to force the fermentation broth from the second fermentation tank into the third fermentation tank via the fluid pathway.

16. The process of claim 15, wherein step b) further comprises supplying gas from the external gas source to a fourth fermentation tank.

17. The process of claim 16, wherein step c) further comprises: during step b), venting gas from the second fermentation tank into the fourth fermentation tank via the fluid pathway, and venting gas from the fourth fermentation tank via a vent of the fourth fermentation tank.

18. The process of claim 17, further comprising:
f) during step b) and after step e), closing the vent of the third fermentation tank, and opening the vent of the fourth fermentation tank, to force the fermentation broth from the third fermentation tank into the fourth fermentation tank via the fluid pathway.

19. A fermentation process comprising:
a) supplying a first fermentation tank with a fermentation broth;
b) supplying gas from an external gas source to a gas inlet at a lower portion of the first fermentation tank and to a gas inlet at a lower portion of a second fermentation tank;
c) during step b), venting gas from a bottom port of the second fermentation tank into a bottom port of the first fermentation tank via a fluid pathway, and venting gas from the first fermentation tank via a vent at an upper portion of the first fermentation tank;
d) during step b) and after step c), closing the vent of the first fermentation tank, and opening a vent at an upper portion of the second fermentation tank, to force substantially all of the fermentation broth from the first fermentation tank into the second fermentation tank, via the bottom port of the first fermentation tank, the fluid pathway, and the bottom port of the second fermentation tank; and
e) during step b) and after step d), closing the vent of the second fermentation tank, and opening the vent of the first fermentation tank, to force substantially all of the fermentation broth from the second fermentation tank into the first fermentation tank, via the bottom port of the second fermentation tank, the fluid pathway, and the bottom port of the first fermentation tank.

20. A fermentation system comprising:
a) a first fermentation tank and a second fermentation tank, each fermentation tank having, respectively, a lower portion with a gas inlet and a bottom port, and an upper portion with an openable and closeable vent;
b) an external gas source in communication with the gas inlet of the first fermentation tank and the gas inlet of the second fermentation tank, for supplying gas to the first fermentation tank and the second fermentation tank; and
c) a fluid pathway extending from the bottom port of the first fermentation tank to the bottom port of the second fermentation tank, for providing fluid communication between the first fermentation tank and the second fermentation tank.

21. The system of claim 20, further comprising a first sparger in the first fermentation tank and serving as the gas inlet of the first fermentation tank, and a second sparger in the second fermentation tank and serving as the gas inlet of the second fermentation tank.

22. The system of claim 20 or claim 21, wherein the vent of the first fermentation tank is at an uppermost portion of the first fermentation tank, and the vent of the second fermentation tank is at an uppermost portion of the second fermentation tank.

23. The system of any one of claims 20 to 22, wherein the bottom port of the first fermentation tank is at a lowermost portion of the first fermentation tank, and the bottom port of the second fermentation tank is at a lowermost portion of the second fermentation tank.

24. The system of any one of claims 20 to 23, wherein the external gas source comprises a compressed air source.

25. The system of any one of claims 20 to 24, further comprising a third fermentation tank, wherein the external gas source and the fluid pathway are in communication with the third fermentation tank.

26. The system of claim 25, further comprising a fourth fermentation tank, wherein the external gas source and the fluid pathway are in communication with the fourth fermentation tank.

27. The system of claim 26, further comprising a set of valves for controlling flow amongst the first, second, third and fourth fermentation tanks via the fluid pathway.
